(19) United States
(12) Patent Application Publication
(10) Pub. No.: US 2015/0289837 A1
(43) Pub. Date: Oct. 15, 2015

(54) ULTRASONIC APPARATUS AND CONTROL METHOD FOR THE SAME

(71) Applicant: SAMSUNG ELECTRONICS CO., LTD., Suwon-si (KR)

(72) Inventors: Baehyung KIM, Yongin-si (KR); Youngil KIM, Suwon-si (KR); Jong Keun SONG, Yongin-si (KR); Seungheun LEE, Seongnam-si (KR); Taeho JEON, Seoul (KR); Kyungil CHO, Seoul (KR)

(73) Assignee: SAMSUNG ELECTRONICS CO., LTD., Suwon-si (KR)

(21) Appl. No.: 14/683,663

(22) Filed: Apr. 10, 2015

(30) Foreign Application Priority Data
Apr. 14, 2014 (KR) 10-2014-0044451

(51) Int. Cl. A61B 8/06; A61B 8/00; A61B 8/08

(52) U.S. Cl. CPC A61B 8/06; A61B 8/5207; A61B 8/488; A61B 8/5223; A61B 8/463; A61B 8/54

(57) ABSTRACT

An ultrasonic apparatus includes a transducer configured to irradiate ultrasonic waves in different traveling directions onto an object and collect echo ultrasonic waves reflected from the object, and a controller configured to determine blood flow velocities of blood flowing in the object based on the echo ultrasonic waves, compound the determined blood flow velocities, and determine a composite blood flow velocity of the blood flowing in the object based on the compounded blood flow velocities.

### CROSS-REFERENCE TO RELATED APPLICATION

This application claims the benefit of Korean Patent Application No. 10-2014-0044451, filed on Apr. 14, 2014 in the Korean Intellectual Property Office, the disclosure of which is incorporated herein by reference in its entirety.

### BACKGROUND

1. Field

Exemplary embodiments relate to an ultrasonic apparatus for imaging an ultrasonic signal and a control method for the same.

2. Description of the Related Art

Ultrasonic diagnostic apparatuses direct ultrasonic signals from a surface of an object (e.g., human body) to a desired region inside the object, and may obtain an image related to a mono layer of soft tissue or a blood-flow using the ultrasonic signals reflected from the desired region, in other words, obtain information of the ultrasonic echo signals in a non-invasive manner.

In general, ultrasonic diagnostic apparatuses have a small size, a low price, a real-time displaying function, and high safety because of no exposure to radiation, such as X-ray radiation. Thus, ultrasonic diagnostic apparatuses are widely used for diagnosis of cardiac disease, breast disease, abdominal disease, urinary system disease, obstetrics and gynecologic disease, and so on.

However, certain conventional ultrasonic diagnostic apparatuses generate ultrasonic images using only magnitudes of reflected ultrasonic signals, thus having difficulty in checking detailed characteristics of a medium into which an ultrasonic wave is directed. Therefore, recently, an ultrasonic functional image, which is an ultrasonic image considering parameters such as elasticity, attenuation, and sound velocity, has been used in addition to a general ultrasonic image.

### SUMMARY

In general, an ultrasonic apparatus for an ultrasonic diagnosis provides a B-mode image of an object. However, in the B-mode image, it is difficult to observe a dynamic organ inside the object, specifically a movement of blood flow. Accordingly, the movement of the blood flow may be displayed on a screen by acquiring a Doppler image as an ultrasonic image.

In this case, the Doppler image is generated using the Doppler effect. As an incident angle, which is an angle between the traveling direction of the blood flow and the irradiation of the ultrasonic wave, becomes closer to 90 degrees, the velocity of the blood flow becomes more difficult to measure.

Accordingly, the exemplary embodiments provide an ultrasonic apparatus that irradiates a plurality of ultrasonic waves having different traveling directions onto an object and compounds the acquired blood flow velocities to acquire a composite blood flow velocity and a control method for the same in order to acquire an accurate blood flow velocity.

In accordance with an aspect of an exemplary embodiment, there is provided an ultrasonic apparatus including a transducer configured to irradiate ultrasonic waves in different traveling directions onto an object and collect echo ultrasonic waves reflected from the object, and a controller configured to determine blood flow velocities of blood flowing in the object based on the echo ultrasonic waves, compound the determined blood flow velocities, and determine a composite blood flow velocity of the blood flowing in the object based on the compounded blood flow velocities.

In accordance with another aspect of an exemplary embodiment, there is provided an ultrasonic apparatus including: a transducer configured to irradiate ultrasonic waves having different traveling directions onto an object and collect echo ultrasonic waves reflected from the object, a controller configured to determine blood flow velocities of blood flowing in the object based on the echo ultrasonic waves, compound the determined blood flow velocities, and determine a composite blood flow velocity of the blood flowing in the object based on the compounded blood flow velocities; and a blood flow image generator configured to generate a blood flow image of the blood flowing in the object based on the composite blood flow velocity of the blood flowing in the object.

In accordance with another aspect of an exemplary embodiment, there is provided a method of controlling an ultrasonic apparatus, the method including: irradiating ultrasonic waves in different traveling directions onto an object; collecting echo ultrasonic waves reflected from the object, determining blood flow velocities of blood flowing in the object based on the collected echo ultrasonic waves; compounding the determined blood flow velocities, and determining a composite blood flow velocity of the blood flowing in the object based on the compounded blood flow velocities.

In accordance with another aspect of an exemplary embodiment, there is provided a method of controlling an ultrasonic apparatus, the method including: irradiating ultrasonic waves in different traveling directions onto an object; collecting echo ultrasonic waves reflected from the object, determining blood flow velocities of blood flowing in the object based on the collected echo ultrasonic waves; compounding the determined blood flow velocities, determining a composite blood flow velocity of the blood flowing in the object based on the compounded blood flow velocities; and generating a blood flow image of the blood flowing in the object based on the composite blood flow velocity.

### BRIEF DESCRIPTION OF THE DRAWINGS

These and/or other aspects of the exemplary embodiments will become apparent and more readily appreciated from the following description of the exemplary embodiments, taken in conjunction with the accompanying drawings of which:

FIG. 1 is a perspective view showing an ultrasonic apparatus according to an exemplary embodiment;

FIG. 2A is a view showing a convex array probe according to an exemplary embodiment, and FIG. 2B is a view showing a linear array probe according to an exemplary embodiment;

FIG. 3 is a block diagram showing a control configuration of an ultrasonic apparatus according to an embodiment of the present invention;

FIG. 4A is a view showing an example of measuring a frequency of a substance inside a stationary object, and FIG. 4B is a view showing an example of measuring a frequency of a substance inside an object moved in a direction indicated by an arrow;

FIG. 5 is a view showing a method of measuring a traveling direction and a velocity of a blood flow using the Doppler effect;

FIG. 6A is a view showing an example of irradiating an ultrasonic wave at an incident angle θ₁ from a transducer, and FIG. 6B is a view showing an example of irradiating an ultrasonic wave at an incident angle θ₂ from a transducer;

FIG. 7A is a view showing a method of a convex array probe irradiating an ultrasonic wave at various irradiation angles according to an exemplary embodiment, and FIG. 7B is a view showing a method of a linear array probe irradiating an ultrasonic wave at various irradiation angles according to an exemplary embodiment;

FIGS. 8A and 8B are views showing a process of acquiring a complex image through compounding according to an exemplary embodiment;

FIG. 9A is a view showing a method of irradiating an ultrasonic wave to acquire volume data of an object according to an exemplary embodiment, and FIG. 9B is a view showing a method of irradiating a plurality of ultrasonic waves onto a plurality of planes to acquire a spatial composite blood flow velocity according to an exemplary embodiment;

FIG. 10 is a view showing an example in which a cross section of an object is used to acquire a planar composite blood flow velocity according to an exemplary embodiment;

FIGS. 11A, 11B, and 11C are views showing a screen displaying an ultrasonic image that is overlaid on a blood flow image in a sequence of time points;

FIG. 12 is a flowchart showing a process of acquiring a composite blood flow velocity according to an exemplary embodiment;

FIG. 13 is a flowchart showing a process of acquiring a planar composite blood flow velocity according to an exemplary embodiment; and

FIG. 14 is a flowchart showing a process of acquiring a spatial composite blood flow velocity according to an exemplary embodiment.

### DETAILED DESCRIPTION

Hereinafter, an ultrasonic apparatus and a control method for the same according to an exemplary embodiment will be described in detail with reference to accompanying drawings.

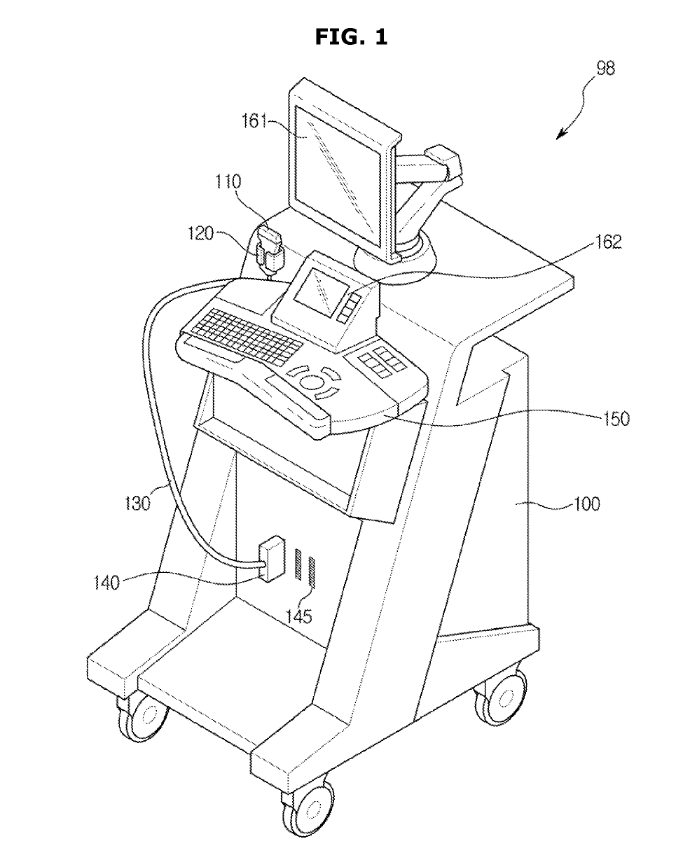

FIG. 1 is a perspective view showing an ultrasonic apparatus 98 according to an exemplary embodiment. As shown in FIG. 1, the ultrasonic apparatus may include a main body 100, an ultrasonic probe 110, an input unit 150, a main display 161, and a sub display 162.

The main body 100 may be provided with at least one female connector 145 at one side. A male connector 140 connected to a cable 130 may be physically coupled to the female connector 145.

The main body 100 may be provided with a plurality of casters on a lower portion thereof to facilitate movement of the ultrasonic apparatus. The casters may be used to fix the ultrasound apparatus to a predetermined position or to move the ultrasound apparatus in a predetermined direction.

The ultrasonic probe 110 is configured to contact a surface of an object and may be configured to transmit and receive an ultrasonic wave. Specifically, the ultrasonic probe 110 irradiates a transmission signal, e.g., an ultrasonic signal, which is provided from the main body 100, inside through the surface of the object (e.g., human body) to an inside thereof, receives an ultrasonic echo signal reflected from a specific portion of the object, and transmits the received ultrasonic echo signal to the main body 100. The cable 130 may have one end connected to the ultrasonic probe 110 and the other end connected to the male connector 140. The male connector 140 connected to the other end of the cable 130 may be physically coupled to the female connector 145 of the main body 100.

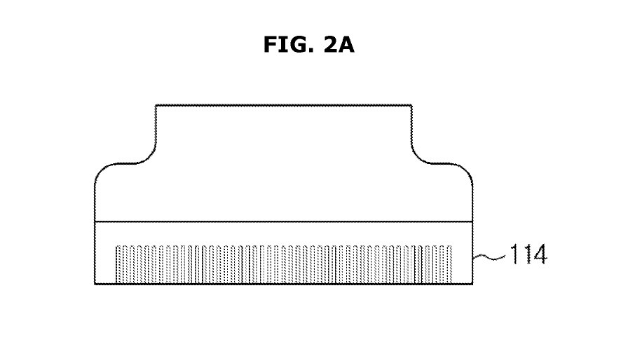

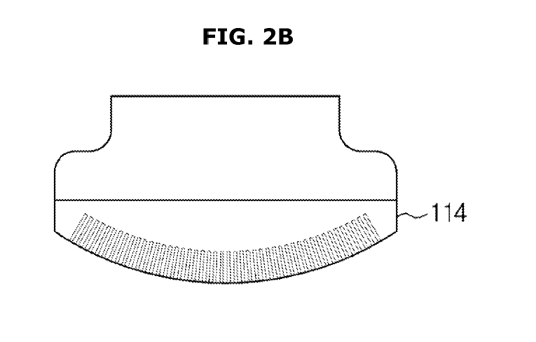

Types of the ultrasonic probe will be described with reference to FIGS. 2A and 2B. FIG. 2A is a convex array probe according to an exemplary embodiment, and FIG. 2B is a linear array probe according to an exemplary embodiment. One or more transducer elements for irradiating and collecting an ultrasonic wave may be provided at a front end of the ultrasonic probe 110. The type of the ultrasonic probe 110 may be classified according to a shape of an array of the transducer elements.

Referring to FIG. 2A, the convex array probe may have transducer elements that are arranged in a curved shape and transmit and receive an ultrasonic wave in directions according to the curved surface. The convex array probe is mainly used for abdominal diagnosis in obstetrics and gynecology (OB/GYN) and may diagnose a deep body part, although is not limited thereto.

In contrast, the linear array probe shown in FIG. 2B has transducer elements that are arranged in a straight line and thus transmit and receive an ultrasonic wave in a forward direction. The linear array probe may be implemented as a high resolution probe because the linear array probe is mainly used to inspect a breast, thyroid gland, blood vessel system, or any other part close to the skin.

The above-described examples of the ultrasonic probe 110, which may be used for the ultrasonic apparatus and the control method for the same according to exemplary embodiments, are merely two examples of an exemplary embodiment, and are not limited to the above examples. Accordingly, in an ultrasonic apparatus and a control method for the same according to another exemplary embodiment, the ultrasonic probe may be a two-dimensional (2D) array probe.

Referring back to FIG. 1, the input unit 150 is configured to receive a command associated with an operation of the ultrasonic apparatus. For example, the input unit 150 may receive a command for selecting a mode such as an amplitude mode (A-mode), a brightness mode (B-mode), and/or a motion mode (M-mode) or a command for initiating ultrasonic diagnosis. The command received through the input unit 150 may be transmitted to the main body 100 over wired or wireless communication.

The input unit 150 may include, for example, at least one of a keyboard, a foot switch, and a foot pedal. The keyboard may be implemented as hardware and positioned on an upper portion of the main body 100. The keyboard may include at least one of a switch, a key, a joystick, and a track ball. Alternatively, the keyboard may be implemented as software such as a graphical user interface. In this regard, the keyboard may be displayed on the main display 161 or the sub display 162. The foot switch or foot pedal may be disposed at a lower portion of the main body 100. The user may control an operation of the ultrasonic apparatus using the foot pedal.

An ultrasonic probe holder 120 for holding the ultrasonic probe 110 may be disposed around the input unit 150. The ultrasonic probe holder 120 may be provided in a plural number. The user may place and contain the ultrasonic probe 110 in the ultrasonic probe holder 120 while the ultrasonic apparatus is not in use.

A display 160 may include the main display 161 and the sub display 162.

The sub display 162 may be disposed at the main body 100. FIG. 1 illustrates that the sub display 162 is disposed on the input unit 150. The sub display 162 may display applications associated with an operation of the ultrasonic apparatus. For example, the sub display 162 may display a menu or instruction required for ultrasonic diagnosis. The sub display 162 may include a cathode ray tube (CRT), a liquid crystal display (LCD), or the like.

The main display 161 may be disposed at the main body 100. In FIG. 1, the main display 161 is disposed over the sub display 162. The main display 161 may display an ultrasonic image obtained during the ultrasonic diagnosis. The main display 161 may include a CRT, an LCD, or the like in the same manner as the sub display 162. FIG. 1 illustrates that the main display 161 is coupled to the main body 100. However, the main display 161 may be detachably disposed on the main body 100.

In FIG. 1, the ultrasonic apparatus is provided with both the main display 161 and the sub display 162. However, the sub display 162 may be omitted if necessary. In this case, the application or menu displayed on the sub display 162 may be displayed on the main display 161.

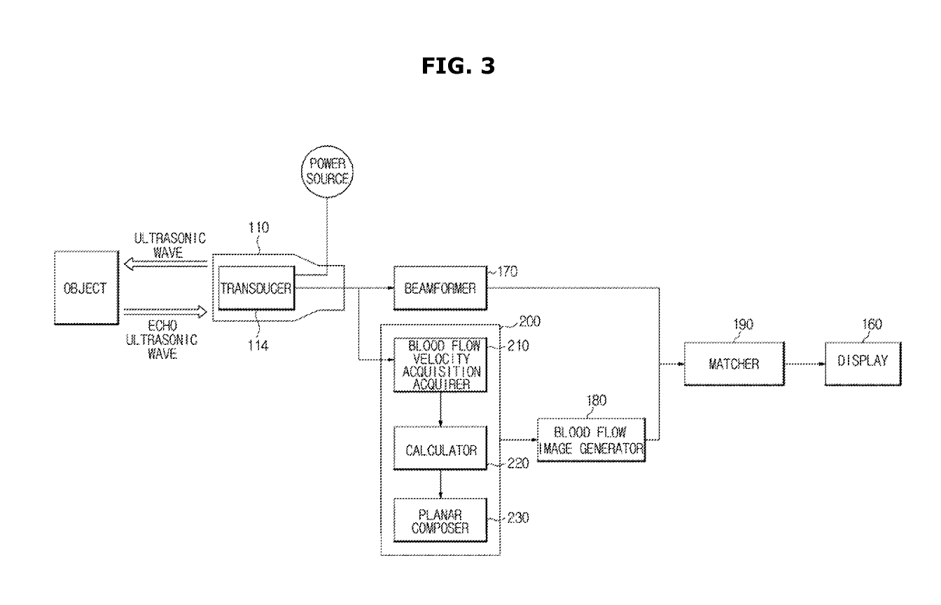

FIG. 3 is a block diagram showing a control configuration of an ultrasonic apparatus according to an exemplary embodiment.

The ultrasonic probe 110 is provided with a plurality of transducers 114. The transducers 114 may generate an ultrasonic pulse according to an alternating current applied from a power source 112, irradiate the ultrasonic pulse onto an object, receive an echo ultrasonic wave reflected from a target part inside the object, and convert the received echo ultrasonic wave into an ultrasonic echo signal, which is an electrical signal. According to an exemplary embodiment, the power source 112 may be an external power supply or an electrical storage device inside the ultrasonic apparatus.

Each of the transducers 114 may be implemented as a magnetostrictive ultrasonic transducer using magnetostriction of a magnetic substance, a piezoelectric ultrasonic transducer using the piezoelectric effect of a piezoelectric material, and a capacitive micromachined ultrasonic transducer (hereinafter simply referred to as a cMUT) transmitting and receiving an ultrasonic wave by using vibrations of hundreds or thousands of micro-processed thin films.

As described above, the ultrasonic probe 110 may be implemented as different types depending on a type or an arrangement of the transducers 114.

When an alternating current is applied from the power source to the transducer 114, a piezoelectric vibrator or a thin film of the transducer 114 is vibrated to generate an ultrasonic pulse. The generated ultrasonic pulse is irradiated onto an object, for example, an object in a human body. The irradiated ultrasonic pulse is reflected by at least one targeted part that is positioned at various depths inside the object. The transducer 114 collects an echo ultrasonic wave, which is the ultrasonic pulse reflected by the target part and returned, and converts the collected echo ultrasonic wave into an ultrasonic echo signal which is an electrical signal.

After converting the echo ultrasonic wave into the ultrasonic echo signal, the transducer 114 may generate an ultrasonic image (B-mode image) based on the ultrasonic echo signal. The generated ultrasonic image may be used to check an inner side of an object during ultrasonic diagnosis.

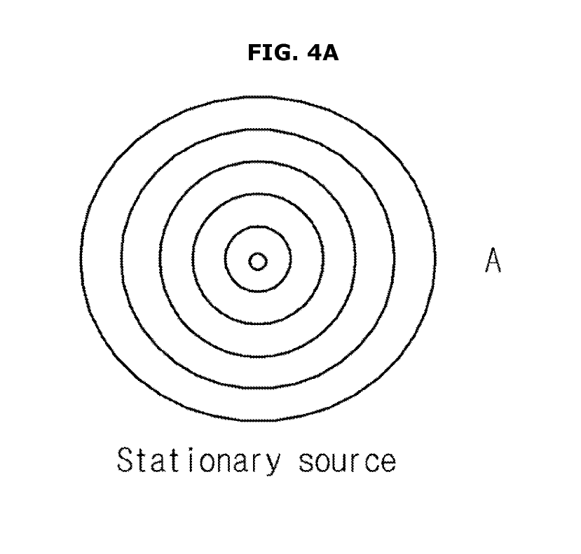

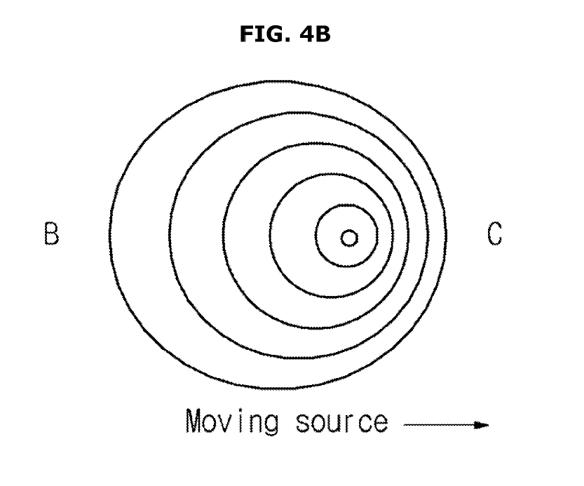

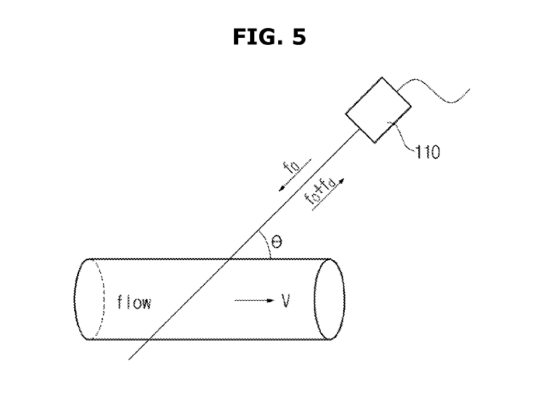

Alternatively, the echo ultrasonic wave may be used to acquire information on a dynamic organ inside the object. A method of acquiring information on a dynamic organ inside an object using the echo ultrasonic wave will be described with reference to FIGS. 4A, 4B, and 5. The dynamic organ may include a blood vessel. Thus the following description will be made on the assumption that the dynamic organ is a blood vessel, although it is understood that the dynamic organ may be many other types of organs.

The Doppler effect may be used to acquire an image of a blood vessel. The Doppler effect is an effect which occurs when at least one of a wave source that generates a wave and an observer that observes the wave is moving. A frequency of the wave becomes higher as the distance between the wave source and the observer is reduced. Conversely, a frequency of the wave becomes lower as the distance between the wave source and the observer is increased.

FIGS. 4A and 4B are views illustrating the Doppler effect which is used in ultrasonic diagnosis. FIG. 4A shows an example of when an ultrasonic probe is positioned at A with respect to a substance inside an object, and FIG. 4B shows an example of when the ultrasonic probe is positioned at B and C with respect to the substance inside the object. In FIGS. 4A and 4B, it is assumed that a frequency of an ultrasonic wave irradiated from the ultrasonic probe onto the substance inside the object is f₀.

As shown in FIGS. 4A and 4B, an ultrasonic wave may be irradiated onto a substance inside a stationary object. As described above, a frequency of the irradiated ultrasonic wave is f₀. When the irradiated ultrasonic wave is reflected by the substance inside the stationary material, an ultrasonic probe positioned at a position A may collect an echo ultrasonic wave reflected by the substance inside the stationary material. In this case, since the substance inside the object is stationary, the Doppler effect does not occur. Accordingly, the collected echo ultrasonic wave has the same frequency as the irradiated infrasonic wave, irrespective of a position of the ultrasonic probe, thereby having the frequency f₀ at the position A.

Unlike in FIG. 4A, the ultrasonic wave having the frequency f₀ may be irradiated onto a substance inside a moving object. The irradiated ultrasonic wave is reflected by the substance inside the object and collected in the form of an echo ultrasonic wave by the ultrasonic probe positioned at positions B and C.

In the case when the substance inside the object moves in a direction indicated by an arrow in FIG. 4B, a wavelength of the echo ultrasonic wave is lengthened in an opposite direction to the direction indicated by the arrow. Accordingly, an ultrasonic probe positioned at the position B collects an echo ultrasonic wave having a frequency less than f₀ which is a frequency of the irradiated ultrasonic wave.

Conversely, the wavelength of the echo ultrasonic wave is compressed and shortened in a direction in which the substance inside the object moves. Accordingly, an ultrasonic probe positioned at the position C, toward which the substance inside the object moves, collects an echo ultrasonic wave having a frequency greater than f₀ which is a frequency of the irradiated ultrasonic wave.

Such an effect may be applied to an ultrasonic diagnosis for blood vessels. The echo ultrasonic wave collected after an ultrasonic wave irradiated from the ultrasonic probe onto the object collides with a red blood cell flowing through a blood vessel and returns has a different frequency from the irradiated ultrasonic wave. The difference may be used to perform imaging.

As described above, when the ultrasonic probe is positioned forward of the direction in which a red blood cell is moving, the frequency of the collected echo ultrasonic wave is higher than that of the irradiated ultrasonic wave. Conversely, if the ultrasonic probe is positioned rearward of the direction in which the red blood cell is moving, the frequency of the collected echo ultrasonic wave is lower than that of the irradiated ultrasonic wave. That is, a traveling direction and a velocity of a blood flow may be checked by comparing the frequency of the echo ultrasonic wave with the frequency of the irradiated ultrasonic wave.

FIG. 5 shows a method of measuring a traveling direction and a velocity of a blood flow using the Doppler effect. In order to check a traveling direction and a velocity of a blood flow flowing through a blood vessel, an ultrasonic wave is irradiated onto the object at an incident angle of θ. In this case, a frequency of the irradiated ultrasonic wave is f₀.

The ultrasonic wave irradiated into the object is reflected by a red blood cell flowing through a blood vessel and collected by an ultrasonic probe. A frequency of the collected echo ultrasonic wave is changed by f_{d} relative to an initial frequency f₀ of the irradiated ultrasonic wave. A frequency variation f_{d} may be calculated using Equation 1 below:

$${f}_{d} = \frac{2 {vf}_{0}}{c} cos θ \qquad [ Equation 1 ]$$

where f_{d} is a frequency variation between an irradiated ultrasonic wave and a collected echo ultrasonic wave, c is an ultrasonic sound velocity, f₀ is a frequency of the irradiated ultrasonic wave, θ is an incident angle that is an angle between an irradiation direction of the ultrasonic wave and a traveling direction of a blood flow, and v is a velocity of the blood flow.

If the frequency f₀ of the irradiated ultrasonic wave, the frequency variation f_{d} between an irradiated ultrasonic wave and a collected echo ultrasonic wave, the ultrasonic sound velocity c, and the incident angle θ are known, the velocity of the blood flow may be found using Equation 1. In this case, a sign of f_{d} may indicate a direction of the blood flow.

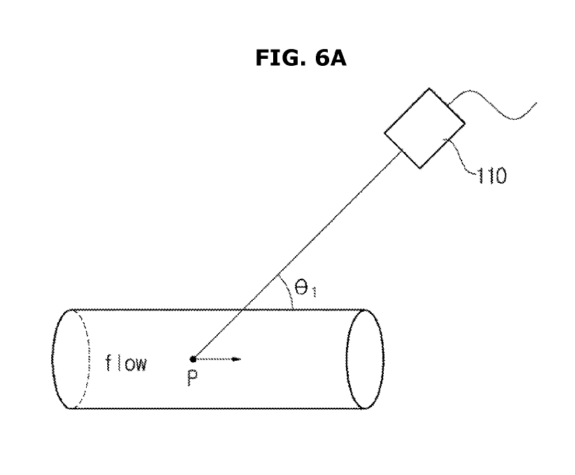

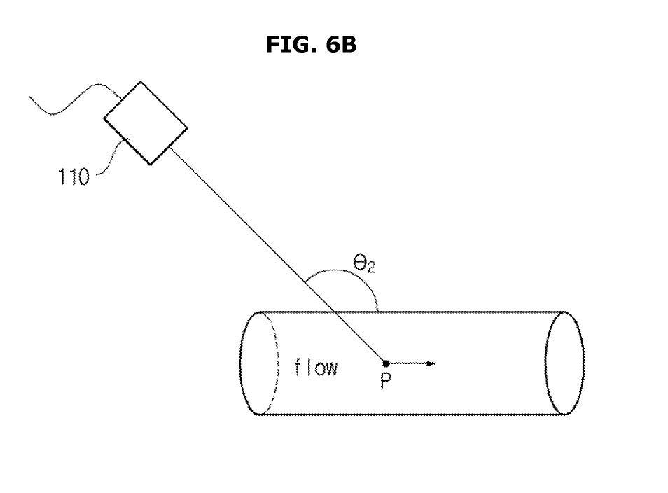

FIG. 6A is a view showing an example of irradiating an ultrasonic wave at an incident angle θ₁ from a transducer. As shown in FIG. 6A, an ultrasonic wave irradiated from the transducer 114 in the ultrasonic probe 110 onto the object travels into a blood vessel at an incident angle θ₁. The ultrasonic wave traveling into the blood vessel may be reflected by a red blood cell inside the blood vessel and collected as an echo ultrasonic wave by the ultrasonic probe. The traveling direction and the velocity of the blood flow may be obtained using Equation 1 on the basis of frequencies of the irradiated ultrasonic wave and the reflected echo ultrasonic wave.

However, when the Doppler effect is considered to acquire the traveling direction and velocity of the blood flow, the acquired information may vary depending on the angle θ between the irradiation direction of the ultrasonic wave and the traveling direction of the blood flow. Specifically, as seen from Equation 1, when θ increases, cos θ decreases and thus the frequency variation f_{d} decreases. When the frequency variation f_{d} decreases, the acquired blood flow velocity may decrease, thereby making the accurate blood flow information difficult to obtain.

To solve this problem, a method of acquiring a composite blood flow velocity based on a plurality of blood flow velocities may be used. Since the plurality of blood flow velocities should be acquired in order to acquire the composite blood flow velocity, a plurality of ultrasonic waves having different incident angles should be irradiated onto the object. A method of an ultrasonic probe irradiating a plurality of ultrasonic waves having different traveling directions will be described below.

FIG. 6B is a view showing an example of irradiating ultrasonic waves at incident angles θ₂ from an ultrasonic probe 110. When an ultrasonic wave is irradiated onto an object in several directions rather than in only one direction, an incident angle, which is an angle between the traveling direction of the blood flow and the irradiation direction of the ultrasonic wave, may be changed. When the traveling direction and velocity of the blood flow are obtained using Equation 1 for each of the various incident angles, a plurality of blood flow velocities for the incident angles may be obtained. The acquired plurality of blood flow velocities may be used to acquire one composite blood flow velocity.

As shown in FIGS. 6A and 6B, ultrasonic waves having incident angles θ₁ and θ₂ with respect to a common point P may be irradiated. A blood flow velocity may be acquired for each incident angle using a frequency difference between the irradiated ultrasonic wave and the reflected echo ultrasonic wave. A composite blood flow velocity may be obtained based on the obtained plurality of blood flow velocities. The composite blood flow velocity may have a higher accuracy than a blood flow velocity that is acquired using an ultrasonic wave irradiated in only one direction. In order to acquire the composite blood flow velocity, the plurality of blood flow velocities may be composed through a compounding process.

As described above, the ultrasonic probe 110 may be implemented as different types depending on a type or an arrangement of the transducers 114 installed in the ultrasonic probe 110. A method of irradiating a plurality of ultrasonic waves having different traveling directions from the ultrasonic probe 110 may be determined depending on the type of the ultrasonic probe 110.

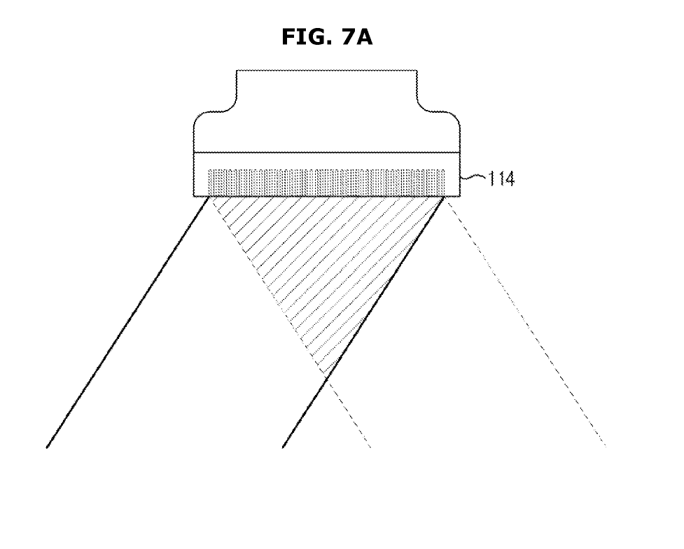

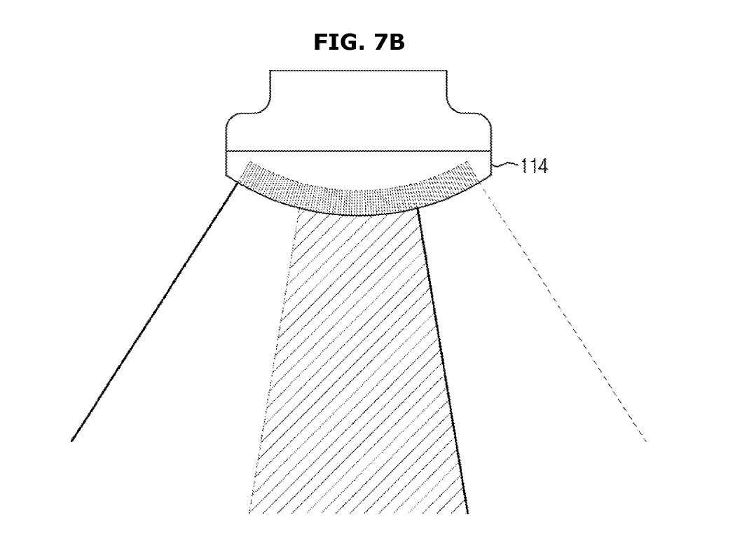

FIG. 7A is a view showing a method of using an ultrasonic probe 110 implemented as a convex array probe to irradiate an ultrasonic wave at various irradiation angles according to an exemplary embodiment, and FIG. 7B is a view showing a method of using an ultrasonic probe 110 implemented as a linear array probe 110 to irradiate an ultrasonic wave at various irradiation angles according to an exemplary embodiment. A solid line and a dashed line indicate ultrasonic waves that are irradiated from the transducers 114 and that have different traveling directions, respectively.

In a convex array probe, since the transducers 144 are arranged along a curved surface, an ultrasonic wave travels in the direction shown in FIG. 7A. Accordingly, a plurality of ultrasonic waves having different traveling directions may be irradiated by mechanically moving a position of the ultrasonic probe 110. Referring to FIG. 7A, ultrasonic waves may be irradiated in a solid-line direction and a dotted-line direction, and in this case, a compounding process may be performed on an overlapping region.

Unlike the convex array probe, the linear array probe may generate only an ultrasonic wave traveling directly forward. Therefore, in order to change a traveling direction of the irradiated ultrasonic wave, the ultrasonic wave may be steered to another direction through electronic calculation.

Each element of the linear array probe performs focusing with its own delay upon irradiating an ultrasonic wave. When the focusing is controlled such that a plurality of elements may have symmetrical delays with respect to the center of the elements, an element positioned at the center appears to irradiate the ultrasonic wave, which is called a scan line. If some elements have asymmetrical delays, the scan line is formed at a certain angle. This asymmetrical delay may have the same effect as an ultrasonic wave being bent and then irradiated in an opposite direction. In FIG. 6B, respective ultrasonic waves travel along the solid line and dashed line by steering the linear array probe, and compounding may be performed on an overlapping portion.

Alternatively, a two-dimensional (2D) array probe enables ultrasonic waves to be irradiated in more directions than the ultrasonic waves irradiated by the ultrasonic probes 110 shown in FIGS. 6A and 6B. Specifically, a one-dimensional (1D) array probe such as a convex array probe or linear array probe allows irradiation of only an ultrasonic wave traveling in the same plane, whereas a 2D array probe in which elements are arranged in 2D allows irradiation of ultrasonic waves traveling in different planes, thereby enabling the traveling direction of the ultrasonic wave to be expressed as a 3D vector. A spatial composite blood flow velocity may be acquired from an object by using such a characteristic, which will be described below.

When ultrasonic waves having different traveling directions are irradiated onto an object, echo ultrasonic waves corresponding to the ultrasonic waves may be acquired. In this case, an irradiation time of the ultrasonic wave and a collection time of the echo ultrasonic wave may be delivered to the main body 100 through a wired or wireless communication network.

Referring back to FIG. 3, a controller 200 may acquire a plurality of blood flow velocities based on incident angles of the plurality of ultrasonic waves having different traveling directions, and compound the acquired plurality of blood flow velocities to acquire a composite blood flow velocity. In this case, the blood flow velocity may be acquired using the delivered frequencies of the irradiated ultrasonic wave and the collected echo ultrasonic wave that are delivered from the transducer 114. However, such a method is merely an example of a method of acquiring the blood flow velocity according to an exemplary embodiment, but is not limited to the above example.

Specifically, the controller 200 may include a blood flow velocity acquisition unit 210 (e.g., blood flow velocity acquisition acquirer) configured to acquire a blood flow velocity for each incident angle from a frequency difference between the irradiated ultrasonic wave and the collected echo ultrasonic wave and a calculation unit 220 (e.g., calculator) configured to compound the acquired plurality of blood flow velocities according to a compounding algorithm to acquire a composite blood flow velocity. Furthermore, when the composite blood flow velocity acquired by the calculation unit 220 is a planar composite blood flow velocity, the controller 200 may include a planar composition unit 230 (e.g., planar composer) configured to compound planar composite blood flow velocities to acquire a spatial composite blood flow velocity.

The blood flow velocity acquisition unit 210 may use frequencies of the irradiated ultrasonic wave and the collected echo ultrasonic wave in order to acquire a blood flow velocity of an object. As described above, the Doppler effect may be used to find the blood flow velocity using Equation 1. Accordingly, the blood flow velocity may be acquired based on a frequency of an irradiated ultrasonic wave, an incident angle, a sound velocity of the ultrasonic wave, and a frequency difference between the ultrasonic wave and an echo ultrasonic wave.

Different traveling directions of the irradiated ultrasonic waves denote different incident angles, which are angles between the traveling direction of the blood flow and the irradiation directions of the ultrasonic waves. Accordingly, frequencies of the echo ultrasonic waves are different, corresponding to the different incident angles, and a plurality of blood flow velocities obtained using Equation 1 on the basis of the different frequencies may be different from one another. That is, the blood flow velocities of the object that are obtained according to the traveling directions of the irradiated ultrasonic waves may be different.

The calculation unit 220 may compound the plurality of blood flow velocities acquired according to the traveling direction of the ultrasonic wave to acquire a composite blood flow velocity. In this case, the compounding may be performed based on a compounding algorithm that is previously stored or inputted by a user or internal calculation.

The compounding process is an ultrasonic wave technique for combining several screens obtained at different angles to acquire one complex image. With this technique, it is possible to reduce an artifact of an image to increase an image quality, compared to an existing ultrasonic technique. It is also possible to quantitatively reduce a speckle noise in the complex image, thus facilitating discovery of a lesion, especially when the contrast is low, and determination of a boundary of the lesion. Accordingly, the enhanced complex image may be obtained by suppressing artifacts such as a speckle noise.

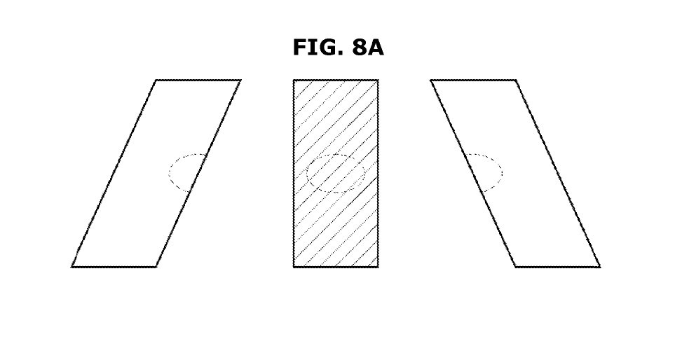

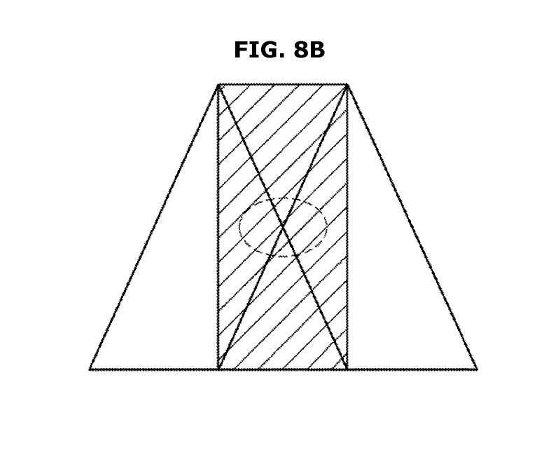

FIGS. 8A and 8B are views showing a process of acquiring a complex image through compounding according to an exemplary embodiment. As shown in FIG. 8A, ultrasonic waves having different directions are irradiated onto an object, and thus a plurality of ultrasonic images are acquired. The left image is acquired by irradiating an ultrasonic wave at a 45 degree angle to the left, the middle image is acquired by irradiating an ultrasonic wave directly forward, and the right image is acquired by irradiating an ultrasonic wave at a 45 degree angle to the right. A dashed-circle or part thereof denotes an object in each ultrasonic image. It can be seen that a figure of an object shown in an ultrasonic image is changed with respect to a traveling direction of the ultrasonic wave. FIG. 8A illustrates an example in which the ultrasonic waves are irradiated in three directions. However, the number of the irradiation directions is not limited thereto, and may be two or more than three.

FIG. 8B shows a process of compounding a plurality of ultrasonic images acquired as shown in FIG. 8A to acquire a composite image. When respective images are adjusted to overlap in consideration of steering angles, an overlapping region corresponds to the composite image. A shaded area in FIG. 8B is an image overlapping region in FIG. 8A, which indicates the composite image. A pixel value in the overlapping region may be determined by a compounding algorithm.

The compounding technique is applicable to an ultrasonic parametric image obtained by imaging detailed characteristics of an object in addition to a general ultrasonic image. Recently, research has been conducted on a technique for applying the compounding technique to ultrasonic elastography.

The compounding technique may be applied to a blood flow velocity which is a critical parameter in ultrasonic diagnosis. The calculation unit 220 may receive the plurality of blood flow velocities acquired based on a plurality of incident angles from the blood flow velocity acquisition unit 210 and compound the plurality of blood flow velocities to acquire the composite blood flow velocity.

The calculation unit 220 may perform the compounding technique according to a compounding algorithm to acquire the composite blood flow velocity. The result may be changed according to the compounding algorithm, which may be selected by a command input through a user or an internal calculation. A mean algorithm, a median filtering algorithm, a root mean square algorithm, a maximum algorithm, and a minimum algorithm will be described below as exemplary embodiments of the compounding algorithm.

The mean algorithm (or the linear average algorithm) is a compound algorithm, which is the most common and widely used in current medical devices. For example, All N number of values of A are added and then divided by N. The mean algorithm is calculated using Equation 2 below:

$${comp}_{mean} = \frac{{A}_{1} + {A}_{2} + {A}_{3} + … + {A}_{N}}{N} \qquad [ Equation 2 ]$$

where compₘₑₐₙ is a composite blood flow velocity at a specific position of an object, A is a blood flow velocity in the object according to a traveling direction of an ultrasonic wave, and N is the number of acquired blood flow velocities.

The median filtering algorithm is a filtering technique for smoothing all values with reference to ambient values. When values in a specific region are aligned in order of size, a median is an output value. A one-dimensional (1D) median filter is applied to the plurality of blood flow velocities. The median filtering algorithm is calculated using Equation 3 below:

comp_{median}=median(A₁,A₂,A₃, . . . , A_{N}) [Equation 3]

where comp_{median} is a composite blood flow velocity at a specific position of an object, A is a blood flow velocity in the object according to a traveling direction of an ultrasonic wave, and N is the number of acquired blood flow velocities.

The root mean square algorithm may assign a weight to the magnitude of the blood flow velocity by using the square of the blood flow velocity. The root mean square algorithm is calculated using Equation 4 below:

$${comp}_{rms} = \frac{\sqrt{( {A}_{1}^{2} + {A}_{2}^{2} + {A}_{3}^{2} + … + {A}_{N}^{2} )}}{N} \qquad [ Equation 4 ]$$

where compᵣₘₛ is a composite blood flow velocity at a specific position of an object, A is a blood flow velocity in the object according to a traveling direction of an ultrasonic wave, and N is the number of acquired blood flow velocities.

The maximum algorithm compares blood flow velocities and determines the maximum blood flow velocity as the composite blood flow velocity. The maximum algorithm is calculated using Equation 5 below:

compₘₐₓ=max(A₁,A₂,A₃, . . . , A_{N}) [Equation 5]

where compₘₐₓ is a composite blood flow velocity at a specific position of an object, A is a blood flow velocity in the object according to a traveling direction of an ultrasonic wave, and N is the number of acquired blood flow velocities.

The minimum algorithm compares blood flow velocities and determines the minimum blood flow velocity as the composite blood flow velocity. The minimum algorithm is calculated using Equation 6 below:

compₘᵢₙ=min(A₁,A₂,A₃, . . . , A_{N}) [Equation 6]

where compₘᵢₙ is a composite blood flow velocity at a specific position of an object, A is a blood flow velocity in the object according to a traveling direction of an ultrasonic wave, and N is the number of acquired blood flow velocities.

Another compounding algorithm may be used in addition to the above-described compounding algorithms. The ultrasonic apparatus and the control method for the same according to exemplary embodiments are not limited to any particular compounding algorithms.

The calculation unit 220 may compound a plurality of blood flow velocities to acquire a composite blood flow velocity. In this case, the composite blood flow velocity may be a planar composite blood flow velocity. When the transducers 114 are arranged in one dimension (for example, in a z-axis direction), an ultrasonic wave irradiated by each of the transducers 114 travels in the same plane (for example, x-z plane). In addition, since steering is performed along a direction (z axis) in which the transducers 114 are arranged, ultrasonic waves irradiated before and after the steering travel in the same plane (for example, x-z plane) although directions in which the ultrasonic waves travel are different. Accordingly, information on a cross section of an object in the plane (for example, x-z plane) in which the ultrasonic waves travel may be obtained. In this case, the information may include blood flow velocities. Hereinafter, the acquired blood flow velocities are each referred to as a planar composite blood flow velocity.

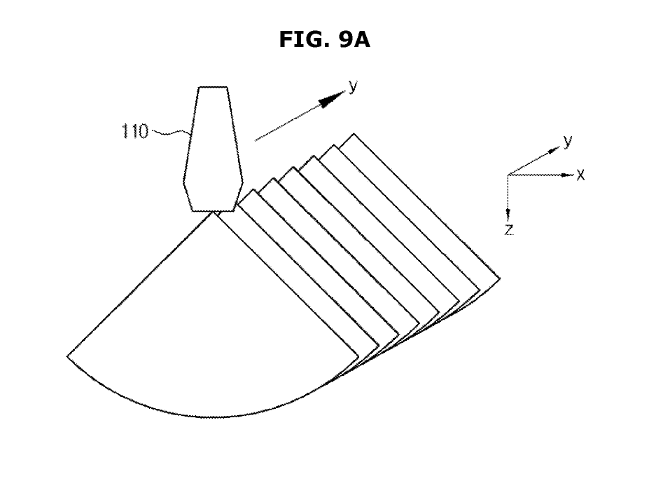

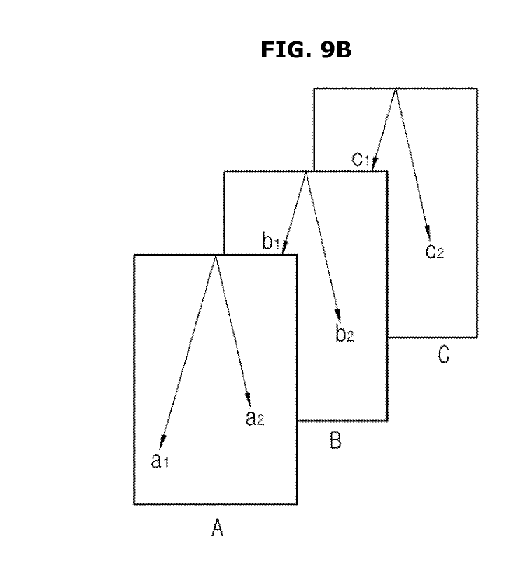

The planar composition unit 230 may compound the acquired planar composite blood flow velocity to acquire a spatial composite blood flow velocity. FIG. 9A is a view showing a method of irradiating an ultrasonic wave to acquire volume data of an object according to an exemplary embodiment. As shown in FIG. 9A, in general, the volume data of the object is obtained by acquiring and combining information on a plurality of cross sections. While the ultrasonic probe 110 is moved in a direction (y-axis) perpendicular to the cross sections, information on each of the cross sections is acquired and added to acquire volume data.

When desired volume data indicates a blood flow velocity in an object, the volume data may be obtained by irradiating a plurality of ultrasonic waves that travel in different directions in different planes to the object and classifying a plurality of blood flow velocities in an object, corresponding to each plane. If the classified blood flow velocities are compounded, a planer composite blood flow velocity in the object may be acquired for each plane. Furthermore, a spatial composite blood flow velocity in the object may be obtained by compounding the planar composite blood flow velocities acquired for the planes.

Specifically, referring to FIG. 9B, a plurality of ultrasonic waves a1, a2, b1, b2, c1, and c2 which travel in different directions in a plurality of planes A, B, and C may be irradiated onto an object. In this case, it is assumed that a1 and a2, which travel in the plane A, are included in an ultrasonic group A, b1 and b2, which travel in the plane B, are included in an ultrasonic group B, and c1 and c2, which travel in the plane C, are included in an ultrasonic group C.

First, blood flow velocities of the object corresponding to ultrasonic waves in each group are acquired. That is, a blood flow velocity of an object corresponding to the ultrasonic group A (a1 and a2), which travels in the plane A, is acquired, a blood flow velocity of the ultrasonic group B (b1 and b2), which travels in the plane B, is acquired, and a blood flow velocity of the ultrasonic group C (c1 and c2), which travels in the plane C, is acquired.

Blood flow velocities are classified and acquired for each group, and then the plurality of blood flow velocities in the same group are compounded. Accordingly, blood flow velocities of the ultrasonic waves a1 and a2 in the ultrasonic group A are compounded. A result (a) of the compounding of the blood flow velocities of the ultrasonic waves a1 and a2 indicates a planar composite blood flow velocity in an object corresponding to the plane A. Similarly, a planar composite blood flow velocity (b) in the object corresponding to the plane B may be acquired by compounding the blood flow velocities of the ultrasonic waves b1 and b2, and a planar composite blood flow velocity (c) in the object corresponding to the plane C may be acquired by compounding the blood flow velocities of the ultrasonic waves c1 and c2.

A spatial composite blood flow velocity may be acquired based on the planar composite blood flow velocities acquired through the above process. In FIG. 9B, a spatial composite blood flow velocity in an object may be acquired by compounding planar blood flow velocities a, b, and c in an object corresponding to the planes A, B, and C.

Unlike in FIGS. 9A and 9B, which show that a planar composite blood flow velocity is acquired while moving the ultrasonic probe 110 in a direction (y-axis) perpendicular to the cross sections, the cross sections used to acquire the planar composite blood flow velocity may intersect with one another. In this case, an intersection region may be in the form of a straight line, and the planar composition unit 230 may perform the compounding on the intersection region according to the above-described compounding algorithms. A spatial composite blood flow velocity may also be found through such a method.

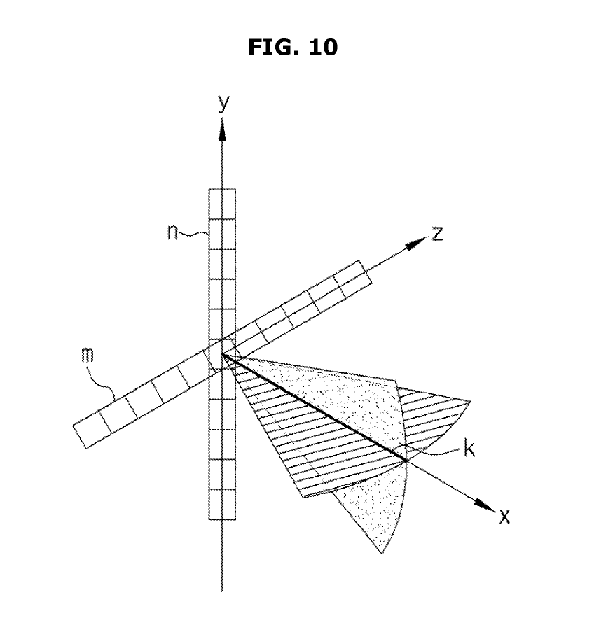

FIG. 10 is a view showing an example in which a vertical cross section of an object is used to acquire a planar composite blood flow velocity according to an exemplary embodiment. First, an element arrangement direction of the transducers 114 may be adjusted to a z-axis and then an ultrasonic wave may be irradiated (operation m). A planar composite blood flow velocity in a cross section of an object in an x-z plane may be acquired based on the irradiated ultrasonic wave. Subsequently, the element arrangement direction of the transducers 114 may be adjusted to a y-axis and then an ultrasonic wave may be irradiated (operation n). As a result, a planar composite blood flow velocity in a cross section of an object in an x-y plane may be acquired. In this case, an intersection region k is formed along an x-axis, and a blood flow velocity therein may be acquired by compounding the planar composite blood flow velocities for the cross sections.

FIG. 10 shows that a planar composite blood flow velocity in a vertical cross section of an object is acquired when the transducers are arranged in one dimension. However, the transducers may be arranged in two dimensions. In this case, a spatial composite blood flow velocity may be acquired by electrically irradiating a plurality of ultrasonic waves traveling in different planes onto an object while an ultrasonic probe is not physically moved.

Since the planar composite blood flow velocity is acquired by irradiating ultrasonic waves traveling in the same plane, the planar composite blood flow velocity may be expressed as a two-dimensional (2D) vector. However, the object is actually in the three-dimensional (3D) form, and thus the blood flow velocity should be expressed as a 3D vector in order to have a more accurate value. As such, the blood flow velocity expressed as the 3D vector may be acquired by compounding planar composite blood flow velocities into a spatial composite blood flow velocity. The acquired blood flow velocity may be closer to an actual blood flow velocity, compared to the blood flow velocity that is expressed as a 2D vector.

Referring back to FIG. 3, the blood flow image generator 180 may generate a blood flow image for an object on the basis of the composite blood flow velocity acquired through the above process. The user may easily diagnose heart disease and cardiovascular disease of an object using the generated blood flow image.

A blood flow image that may be generated based on a traveling direction and a velocity of the blood flow may include a spectral Doppler image, a color flow image, a 3D flow image.

The spectral Doppler image is an image that is generated on the basis of blood flow information measured at a point of interest. With the Doppler effect, a variation of a blood flow over time may be displayed on a screen. The variation may be displayed as a Doppler spectrum on the screen on the basis of information on a measured velocity and direction of the blood flow.

The color flow image may show a planar blood flow in the form of a tomographic image. In the color flow image, for example, a color indicates a traveling direction of the blood flow, and a brightness indicates a velocity of the blood flow. An average velocity, a direction, turbulence, and so on may be displayed in different colors in the color flow image.

The 3D flow image is an image representing the blood flow in a volume of an object, unlike the color flow image representing a tomographic image of an object. As described above, the 3D flow image may be generated using a spatial composite blood flow velocity in the object. Specifically, the 3D flow image may be generated because there is a spatial composite blood flow velocity for each point inside the object and the spatial composite blood flow velocity can be expressed as a 3D vector. The blood flow may be represented in gray-scale or many other types of color representation formats.

The above-described flow images may be video images. Accordingly, a user may check the traveling direction and velocity of the blood flow in real time and also may check the presence of cardiovascular disease in a short time.

Exemplary embodiments of the flow image have been described above. However, the flow image in the ultrasonic apparatus and the control method for the same are not limited thereto, and may include an image generated based on the information on the traveling direction and velocity of the blood flow.

Referring back to FIG. 3, a beamformer 170 may focus echo ultrasonic waves delivered from a transducer and generate an ultrasonic image based on the focused echo ultrasonic waves. In this case, the generated ultrasonic image is based on information acquired according to a degree of reflection of the ultrasonic wave based on a medium inside an object. The image generated by the beamformer 170 may be a B-mode image.

Alternatively, the image generated by the beamformer 170 may be a composite ultrasonic image. Since ultrasonic waves having different traveling directions are irradiated onto an object in order to obtain a composite blood flow velocity, a plurality of echo ultrasonic waves may be acquired. The beamformer 170 may compound a plurality of ultrasonic images converted based on the plurality of echo ultrasonic waves to acquire a composite ultrasonic image. The acquired composite ultrasonic wave may reduce an artifact, thus improving resolution.

The matching unit 190 may receive a blood flow image from the blood flow image generator 180, find a corresponding point of an object corresponding to the blood flow image, and find a corresponding pixel representing the corresponding point in an ultrasonic image received from the beamformer 170. That is, the matching unit 190 may check which point of an object corresponds to the blood flow image and find a pixel representing the checked point in an ultrasonic image. Through this process, the blood flow image may correspond to a specific pixel of the ultrasonic wave on a one-to-one basis.

The display 160 may receive information on the corresponding pixel from the matching unit, overlay the blood flow image on the ultrasonic image based on the received information on the corresponding pixel, and display the overlay image on a screen. If the blood flow image corresponds to a specific point of a blood vessel and the corresponding point corresponds to a specific pixel of an ultrasonic image, the blood flow image may be overlaid on the ultrasonic image by displaying the blood flow image at a position of the corresponding pixel.

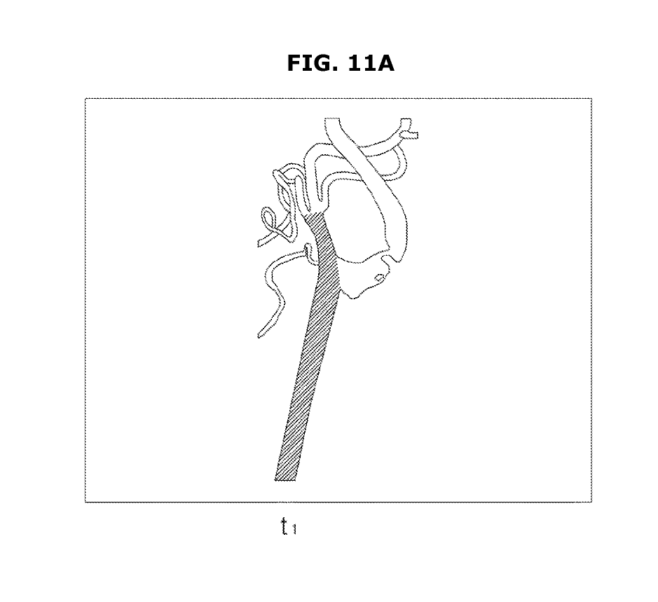

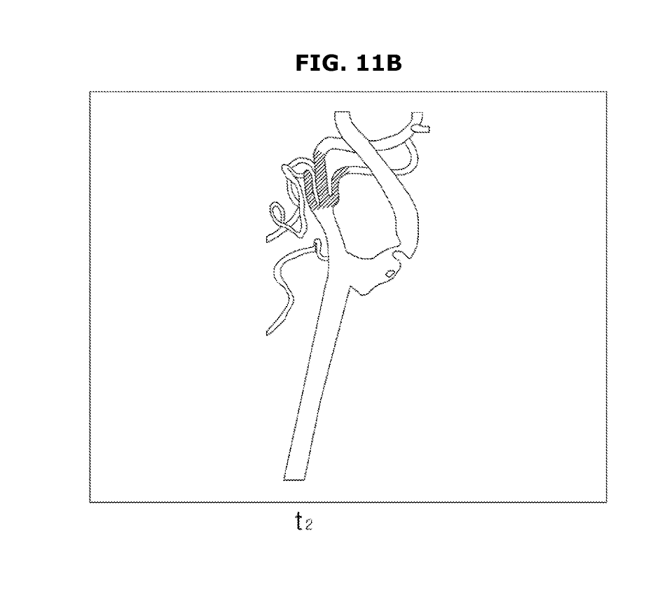

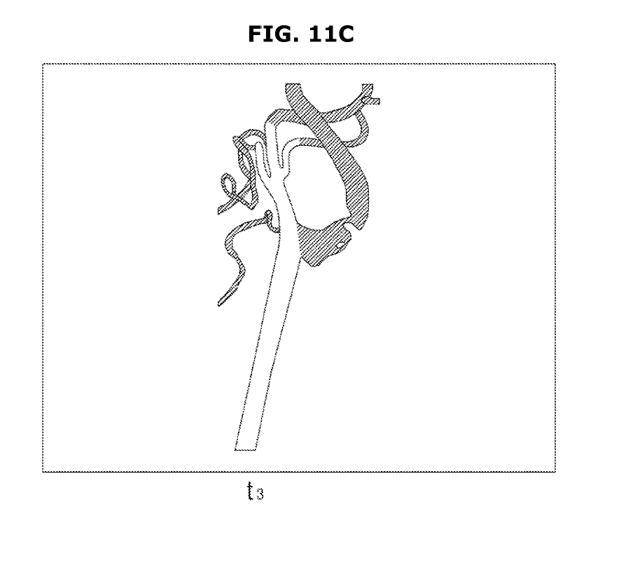

When the blood flow images are used to generate video images, the video images displayed on the screen may be related to a blood flow moving in a blood vessel. FIGS. 11A, 11B, and 11C are views showing a screen displaying a blood flow image that is overlaid on an ultrasonic image as a sequence of time points t₁, t₂, and t₃.

As described above, the ultrasonic image may be acquired using a characteristic in which a reflectance of an ultrasonic wave varies depending on a medium inside an object. Accordingly, the ultrasonic image may include information about an internal structure of the object. The internal structure of the object may be in the form of a blood vessel.

When blood flow images varying over time are acquired in the form of video images, it is possible to check a position in the blood vessel where the blood flows for each frame of the blood flow image and to display the blood flow in a pixel representing a point of a blood vessel where a blood flows in the ultrasonic image.

For example, as shown in FIGS. 11A, 11B, and 11C, the form of the blood vessel may be acquired through the ultrasonic image and remain constant irrespective of time. The blood flow may be overlaid on a pixel representing a blood vessel of the ultrasonic image. Since the blood flows through the blood vessel over time, the blood flow may be displayed on a screen of the display 160 such that the blood flow may be observed at different positions of the blood vessel.

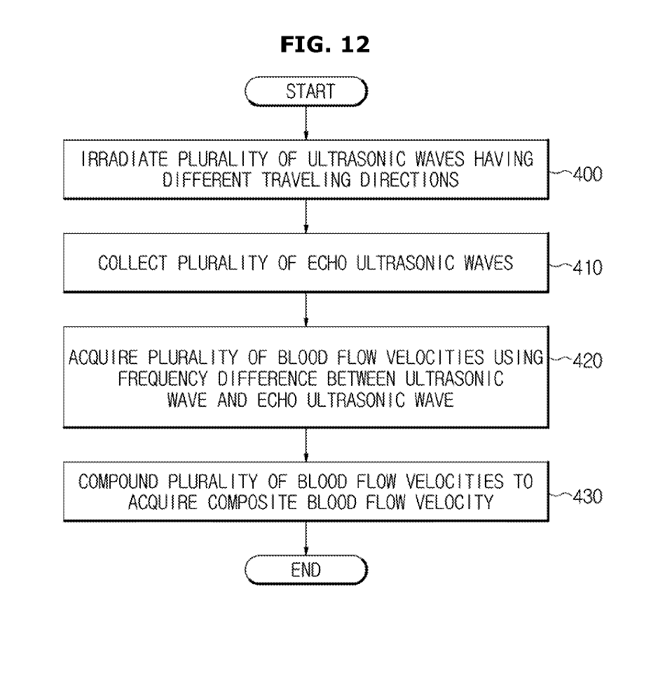

FIG. 12 is a flowchart showing a process of acquiring a composite blood flow velocity according to an exemplary embodiment.

First, a plurality of ultrasonic waves having different traveling directions are irradiated onto an object in operation 400. A method of irradiating ultrasonic waves having different directions may vary depending on the ultrasonic probe 110. A convex array probe may irradiate the ultrasonic waves while adjusting a steering angle using a mechanical steering method. A linear array probe may irradiate the ultrasonic waves while adjusting a steering angle using an electronic steering method. Alternatively, a two-dimensional (2D) array probe may be used to irradiate the ultrasonic waves having different traveling directions.

The reason the ultrasonic waves are irradiated in different traveling directions is that a method of finding blood flow velocities in several directions to acquire a composite blood flow velocity may be more accurate than a method of measuring a blood flow velocity of an object in only one direction.

After the plurality of ultrasonic waves are irradiated, a plurality of echo ultrasonic waves corresponding thereto may be collected in operation 410. When a frequency of each of the collected echo ultrasonic waves is checked, a blood flow velocity of an object may be acquired based on the checked frequency.

Next, a frequency difference between the irradiated ultrasonic waves and the collected echo ultrasonic waves may be found, and then the blood flow velocity may be acquired based on the frequency difference in operation 420. The frequency difference between the irradiated ultrasonic wave and the collected echo ultrasonic wave may be expressed in an equation for the blood flow velocity. The equation corresponds to Equation 1, described above.

Since the found blood flow velocity is measured based on an ultrasonic wave traveling along one path, a plurality of blood flow velocities corresponding to the traveling directions of the irradiated ultrasonic waves may be acquired.

Lastly, the plurality of blood flow velocities may be compounded to acquire a composite blood flow velocity in operation 430. The difference from an actual blood flow velocity may be reduced by compounding different blood flow velocities at the same point to acquire a composite blood flow velocity.

The compounding may be performed based on a compounding algorithm that is previously stored, inputted by a user, or determined by an internal operation. The types of compounding algorithms include a mean algorithm, a median filtering algorithm, a root mean square algorithm, a maximum algorithm, and a minimum algorithm. However, the compounding algorithms are merely examples and may include any other algorithm that may find a composite blood flow velocity through compounding.

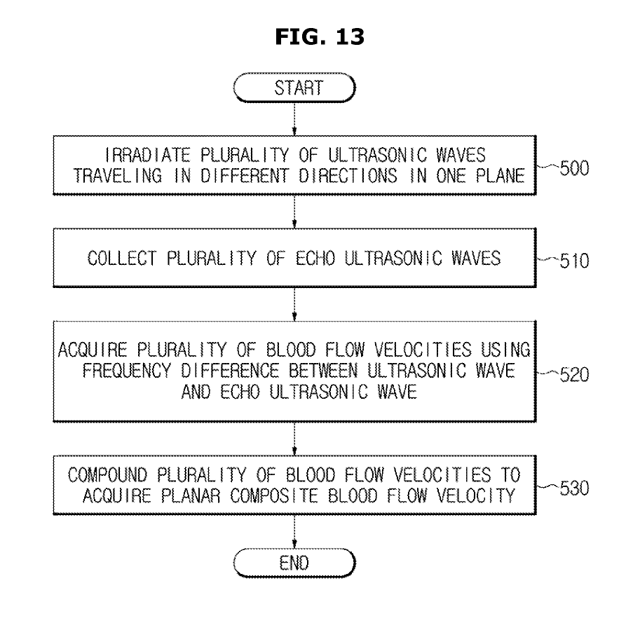

FIG. 13 is a flowchart showing a process of acquiring a planar composite blood flow velocity according to an exemplary embodiment. According to an exemplary embodiment, the planar composite blood flow velocity may be acquired on the assumption that elements of the ultrasonic probe 110 are arranged in one dimension and the irradiated ultrasonic wave is steered in a direction in which the elements are arranged.

First, a plurality of ultrasonic waves that travel in different directions and in the same plane are irradiated onto an object in operation 500. When the irradiated ultrasonic waves are steered to directions according to an arrangement of the transducers 114, the irradiated ultrasonic waves do not have a component perpendicular to the direction in which the transducers 114 are facing, and thus the irradiated ultrasonic waves travel in the same plane.

A plurality of echo ultrasonic waves may be collected corresponding to the irradiated plurality of ultrasonic waves in operation 510. A blood flow velocity may be acquired using a frequency difference between the irradiated ultrasonic waves and the collected echo ultrasonic waves in operation 520. The blood flow velocity in the object is acquired for each incident angle of the irradiated ultrasonic waves.

Lastly, the acquired plurality of blood flow velocities may be compounded to acquire a planar composite blood flow velocity in operation 530. In this case, the acquired planar composite blood flow velocity denotes a blood flow velocity of an object corresponding to a plane in which the plurality of ultrasonic waves travel.

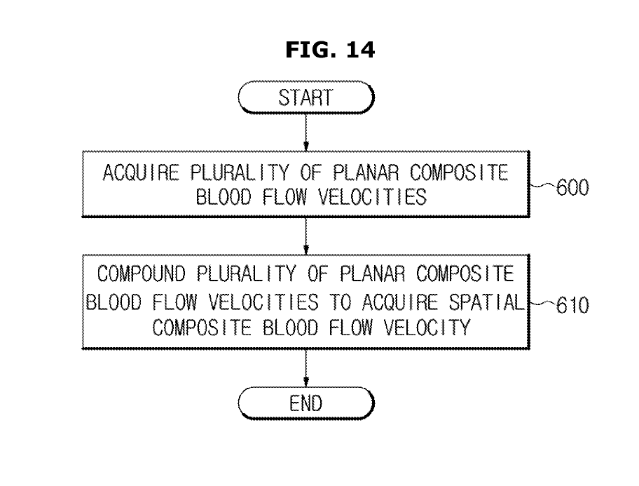

FIG. 14 is a flowchart showing a process of acquiring a spatial composite blood flow velocity according to an exemplary embodiment. When planar composite blood flow velocities are acquired through the process of FIG. 13, a spatial composite blood flow velocity may be acquired based on the planar composite blood flow velocities. There are many methods of acquiring the spatial composite blood flow velocity. However, a method of acquiring planar composite blood flow velocities of the object corresponding to planes intersecting with one another and acquiring the spatial composite blood flow velocity based on the planar composite blood flow velocities will be described below. In this case, it is assumed that planar composite blood flow velocities in an object are acquired corresponding to an x-z plane and an x-y plane.

First, planar composite blood flow velocities in an object are acquired corresponding to a plurality of planes in operation 600. As assumed above, the plurality of planes may intersect with one another. Accordingly, a blood flow velocity in an object corresponding to an x-y plane and a blood flow velocity in an object corresponding to an x-z plane may be acquired.

On the basis of the acquired two blood flow velocities, planar composite blood flow velocities in an internal region of the object, where a plurality of planes intersect with one another, may be compounded to acquire a spatial composite blood flow velocity. That is, in an intersection region on an x-axis, which is a region where an x-y plane and an x-z plane intersect, planar composite blood flow velocities acquired for the planes may be compounded to acquire a spatial composite blood flow velocity. The spatial composite blood flow velocity acquired through the compounding may be represented in three dimensions, thus deriving a more accurate result than the planar composite blood flow velocity.

The ultrasonic apparatus and the control method for the same according to an exemplary embodiment may have the following effects.

According to the ultrasonic apparatus and the control method for the same according to an exemplary embodiment, an accurate blood flow velocity may be measured, thus increasing an accuracy of a blood flow image generated based on the blood flow velocity. Thus, the ultrasonic apparatus and the control method for the same according to an exemplary embodiment may assist a user in diagnosing cardiovascular disease through the blood flow image.

Moreover, according to another exemplary embodiment of the ultrasonic apparatus and the control method for the same, a 3D blood vessel image may be generated and the generated 3D blood vessel image may be overlaid on an ultrasonic image, thus displaying a more accurate image on a screen. On the basis of the image, a user may more accurately diagnose a patient.

Although a few exemplary embodiments have been shown and described, it would be appreciated by those skilled in the art that changes may be made in these exemplary embodiments without departing from the principles and spirit of the exemplary embodiments, the scope of which is defined in the claims and their equivalents.

What is claimed is:

1. An ultrasonic apparatus comprising:
a transducer configured to irradiate ultrasonic waves in different traveling directions onto an object and collect echo ultrasonic waves reflected from the object; and
a controller configured to determine blood flow velocities of blood flowing in the object based on the echo ultrasonic waves, compound the determined blood flow velocities, and determine a composite blood flow velocity of the blood flowing in the object based on the compounded blood flow velocities.

2. The ultrasonic apparatus of claim 1, wherein the controller is configured to compound the determined blood flow velocities based on a mean value of the blood flow velocities at a same point of the object.

3. The ultrasonic apparatus of claim 1, wherein the transducer is configured to irradiate the ultrasonic waves traveling in the different traveling directions onto the object in a single plane, and
wherein the controller is configured to determine a planar composite blood flow velocity of the blood flowing in the object corresponding to the single plane based on the determined blood flow velocities.

4. The ultrasonic apparatus of claim 3, wherein the controller is configured to determine the blood flow velocities of the blood flowing in the object corresponding to the single plane, compound the blood flow velocities, and determine the planar composite blood flow velocity based on the compounded blood flow velocities.

5. The ultrasonic apparatus of claim 1, wherein the transducer is configured to irradiate the ultrasonic waves traveling in the different traveling directions in a plurality of planes, and
the controller is configured to classify and then determine the blood flow velocities of the blood flowing in the object corresponding to the plurality of planes and determine a spatial composite blood flow velocity of the blood flowing in the object based on the classified and then determined blood flow velocities.

6. The ultrasonic apparatus of claim 5, wherein the controller is configured to compound the classified and then determined blood flow velocities, determine planar composite blood flow velocities of the blood flowing in the object corresponding to the plurality of planes based on the compounded blood flow velocities, compound the planar composite blood flow velocities, and determine the spatial composite blood flow velocity of the blood flowing in the object based on the compounded planar composite blood flow velocities.

7. The ultrasonic apparatus of claim 1, wherein the controller is configured to determine the blood flow velocities of the blood flowing in the object using a frequency difference between the irradiated ultrasonic waves and the collected echo ultrasonic waves.

8. An ultrasonic apparatus comprising:
a transducer configured to irradiate ultrasonic waves in different traveling directions onto an object and collect echo ultrasonic waves reflected from the object;
a controller configured to determine blood flow velocities of blood flowing in the object based on the echo ultrasonic waves, compound the determined blood flow velocities, and determine a composite blood flow velocity of the blood flowing in the object based on the compounded blood flow velocities; and
a blood flow image generator configured to generate a blood flow image of the blood flowing in the object based on the composite blood flow velocity of the blood flowing in the object.

9. The ultrasonic apparatus of claim 8, further comprising a matcher configured to identify a pixel representing a point of the object corresponding to the blood flow image in an ultrasonic image acquired based on the echo ultrasonic waves.

10. The ultrasonic apparatus of claim 9, further comprising a display configured to overlay the blood flow image on the ultrasonic image based on the pixel and display the overlayed image on a same screen.

11. The ultrasonic apparatus of claim 9, wherein the ultrasonic image includes a composite ultrasonic image acquired by compounding a plurality of ultrasonic images acquired from the ultrasonic waves having the different traveling directions.

12. A method of controlling an ultrasonic apparatus, the method comprising:
irradiating ultrasonic waves in different traveling directions onto an object;
collecting echo ultrasonic waves reflected from the object;
determining blood flow velocities of blood flowing in the object based on the collected echo ultrasonic waves;
compounding the determined blood flow velocities; and
determining a composite blood flow velocity of the blood flowing in the object based on the compounded blood flow velocities.

13. The method of claim 12, wherein the compounding comprises determining a mean value of the blood flow velocities at a position of the object and performing the compounding based on the determined mean value.

14. The method of claim 12, wherein the irradiating of the ultrasonic waves comprises irradiating the ultrasonic waves in the different traveling directions onto the object in a single plane,
wherein the determining of the blood flow velocities of the blood flowing in the object comprises determining the blood flow velocities of the blood flowing in the object corresponding to the single plane, and
the compounding comprises determining a planar composite blood flow of the blood flowing in the object corresponding to the single plane based on the determined blood flow velocities.

15. The method of claim 12, wherein the irradiating of the ultrasonic waves comprises irradiating the ultrasonic waves traveling in the different traveling directions in a plurality of planes,
wherein the determining of the blood flow velocities comprises classifying and then determining the blood flow velocities of the blood flowing in the object corresponding to the plurality of planes based on the ultrasonic waves respectively traveling in the plurality of planes, and
the compounding comprises determining a spatial composite blood flow velocity of the blood flowing in the object based on the classified and then determined blood flow velocities.

16. The method of claim 15, wherein the compounding comprises:
compounding the classified and then determined blood flow velocities,
determining planar composite blood flow velocities of the blood flowing in the object corresponding to each of the plurality of planes based on the compounded blood flow velocities,
compounding the planar composite blood flow velocities, and
determining the spatial composite blood flow velocity of the blood flowing in the object.

17. A method of controlling an ultrasonic apparatus, the method comprising:
irradiating ultrasonic waves in different traveling directions onto an object;
collecting echo ultrasonic waves reflected from the object;
determining blood flow velocities of blood flowing in the object based on the collected echo ultrasonic waves;
compounding the determined blood flow velocities;
determining a composite blood flow velocity of the blood flowing in the object based on the compounded blood flow velocities; and
generating a blood flow image of the blood flowing in the object based on the composite blood flow velocity.

18. The method of claim 17, further comprising acquiring an ultrasonic image based on the echo ultrasonic waves; and
identifying a pixel representing a point of the object corresponding to the blood flow image of the blood flowing in the ultrasonic image.

19. The method of claim 18, further comprising overlaying the blood flow image on the ultrasonic image based on the pixel and displaying the overlayed image on a same screen.

20. The method of claim 18, wherein the ultrasonic image includes a composite ultrasonic image acquired by compounding a plurality of ultrasonic images acquired from the ultrasonic waves irradiated in the different traveling directions.
